(19) United States
(12) Patent Application Publication
(10) Pub. No.: US 2023/0023513 A1
(43) Pub. Date: Jan. 26, 2023

(54) SELF-SWITCHING TRANSMISSION ASSEMBLY, BALLOON, AND PROSTHESIS FOR USE IN SHOULDER JOINT

(71) Applicant: SHANGHAI ENDOPHIX CO., LTD., Shanghai (CN)

(72) Inventors: Xiaofei JI, Shanghai (CN); Chen LIU, Shanghai (CN); Bin YUE, Shanghai (CN)

(21) Appl. No.: 17/784,517

(22) PCT Filed: Dec. 10, 2020
(86) PCT No.: PCT/CN2020/135270

§ 371 Date: Jun. 10, 2022

(30) Foreign Application Priority Data
Dec. 11, 2019 (CN) 201911268570.8

(51) Int. Cl. A61F 2/46; A61B 17/56; A61F 2/40; A61F 2/48

(52) U.S. Cl. CPC A61F 2/4612; A61B 17/562; A61F 2/40; A61F 2/484; A61F 2002/30584

(57) ABSTRACT

Disclosed are a self-switching transmission assembly, a balloon, and a prosthesis for use in a shoulder joint. The self-switching transmission assembly comprises: a communicating tube (11, 11′, 11″) comprising an input end (111, 111′, 111″) and an output end (112, 112′, 112″); an external connection tube (12, 12′, 12″), an end portion thereof being insertable into the communicating tube (11, 11′, 11″) from the input end (111, 111′, 111″) and removable from the communicating tube (11, 11′, 11″) from the input end (111, 111′, 111″); and a sealing tube (13, 13′, 13″), which can be placed in a tube body of the communicating tube (11, 11′, 11″) and moved axially relative to the tube body of the communicating tube (11, 11′, 11″). The sealing tube (13, 13′, 13″) comprises a connection end (131, 131′, 131″) and a sealing end (132, 132′, 132″), the connection end (131, 131′, 131″) being detachably connected to the end portion of the external connection tube (12, 12′, 12″) by means of a connection driving mechanism, and the sealing end (132, 132′, 132″) being provided with a pass-through region, wherein, when the pass-through region is exposed outside of the output end (112, 112′, 112″), the self-switching transmission assembly is in a pass-through state, and when the pass-through region is placed inside the tube body of the communicating tube (11, 11′, 11″), the self-switching transmission assembly is in a sealed and blocked state.

### CROSS REFERENCE TO RELATED APPLICATION

The present disclosure claims priority to Chinese Patent Application No. 2019112685708, entitled “SELF-SWITCHING TRANSMISSION ASSEMBLY BALLOON, AND PROSTHESIS FOR USE IN SHOULDER JOINT”, filed on Dec. 11, 2019, the content of which is incorporated herein by reference in its entirety.

### TECHNICAL FIELD

The present disclosure relates to the field of medical devices, in particular to a self-on-off transmission assembly, a balloon and a prosthesis for a shoulder joint.

### BACKGROUND

With the aging of the population and the increase in the number of exercising people, the incidence of rotator cuff tear is increasing year by year. At the same time, elderly patients often rarely receive treatment intervention in the early stage due to various reasons such as concept and economic level. Once the disease progresses to the point where surgery is required, it has often developed into a large or irreparable rotator cuff tear, and the treatment will cost more. This not only brings physical pain and economic burden to patients and affects the quality of life of patients, but also gradually becomes a major social health problem.

The rotator cuff supports and stabilizes the scapulohumeral joint during the movement of the shoulder joint. Rotator cuff injury may weaken or even lose the fulcrum function for maintaining the humeral head and the glenoid, which may seriously affect the function of the shoulder joint. When the rotator cuff injury occurs, implantation of a rotator cuff balloon at the joint can be considered. The rotator cuff balloon is mainly used to be implanted between the acromion and the humeral head to ensure a smooth joint movement, reduce friction, and restore shoulder biomechanics.

During the implantation operation, the rotator cuff balloon needs to be folded in a puncture cannula, After being positioned and released under a direct vision of an arthroscope, the balloon is filled by injecting physiological saline through a catheter. After separating the catheter from the balloon, the catheter is withdrawn and the balloon is sealed. Therefore, the design of the rotator cuff balloon needs to ensure that the balloon is disconnected from the catheter without leakage, after being filled. However, it is not easy to separate the rotator cuff balloon, that is currently used clinically, from the catheter, or the filling fluid inside the balloon is easy to flow out from a connection between the rotator cuff balloon and the catheter after the balloon is filled.

### SUMMARY

Accordingly, it is necessary to provide a self-on-off transmission assembly, a balloon and a prosthesis for a shoulder joint, to address problems that the rotator cuff balloon is not easily separated from the catheter and the sealing performance of the rotator cuff balloon is poor.

In an aspect, a self-on-off transmission assembly is provided, and includes: a communication tube, including an input end and an output end; an outer tube, an end of the outer tube being configured to be capable of being inserted into the communication tube from the input end and being moved out of the communication tube from the input end; and a sealing tube configured to be capable of being placed into a tube body of the communication tube and being moved in an axial direction with respect to the tube body of the communication tube, the sealing tube including a connecting end and a sealing end. The connecting end is configured to be detachably connected to the end of the outer tube through a connecting-driving mechanism. The sealing end is provided with a communicating area. When the communicating area is exposed to the output end, the self-on-off transmission assembly is in an on-state; when the communicating area is placed in the tube body of the communication tube, the self-on-off transmission assembly is in a sealed off-state. The outer tube is configured to at least allow the self-on-off transmission assembly to be switchable from being in the on-state to being in the sealed off-state, by driving the sealing tube to move along the communication tube by the connecting-driving mechanism.

Further, the connecting-driving mechanism includes a first member arranged at the end of the outer tube, and a second member arranged at the connecting end of the sealing tube, and the first member and the second member are configured to be capable of being coupled with each other in a circumferential direction and decoupled from each other in the axial direction.

Further, the connecting-driving mechanism includes a third member arranged at the end of the outer tube, and a fourth member arranged at the connecting end of the sealing tube, and the third member and the fourth member are configured to be capable of being coupled with each other in the axial direction and in a circumferential direction, and being decoupled from each other in the axial direction and in the circumferential direction.

Further, the connecting-driving mechanism includes a plug-in cannula arranged at the connecting end of the sealing tube. The plug-in cannula is configured to be capable of being inserted into an inner hole of the end of the outer tube. When the end of the outer tube is inserted into the communication tube, the end of the outer tube is clamped between the communication tube and the plug-in cannula.

Further, the first member includes a first arc-shaped cylinder, the second member includes a second arc-shaped cylinder, and the first arc-shaped cylinder and the second arc-shaped cylinder are capable of being abutted against each other in the circumferential direction.

Further, an outer peripheral wall of the end of the outer tube and an outer peripheral wall of the sealing tube are both provided with outer threads. An inner hole wall of the communication tube is provided with inner threads matched with the outer threads.

Further, the third member is a first buckle, the fourth member is a second buckle, and the first buckle and the second buckle are capable of being engaged with each other.

Further, the first buckle and the second buckle are hooks extending in the circumferential direction.

Further, an inner hole of the communication tube includes a cylindrical hole and a tapered hole which are connected to each other. The tapered hole is close to the output end. The sealing tube includes a cylindrical tube body and an inverted tapered tube body which are connected to each other. The fourth member is arranged on the cylindrical tube body. The communicating area is arranged in the inverted tapered tube body. The cylindrical tube body is slidably matched with the cylindrical hole. The inverted tapered tube body is adapted to be abutted against the tapered hole.

Further, the sealing tube includes a cylindrical tube body and an inverted tapered tube body which are connected to each other. The fourth member and the communicating area are arranged in the cylindrical tube body. The cylindrical tube body is slidably matched with an inner hole of the communication tube. The inverted tapered tube body is adapted to be abutted against an end of the inner hole of the communication tube.

Further, the sealing tube includes: a sealing ring connected to the plug-in cannula, an outer periphery surface of the sealing ring being sealingly fitted with an inner hole wall of the communication tube; an end ring; the communicating area being arranged on an periphery wall of the end ring, an outer periphery surface of the end ring being capable of being sealingly fitted with the inner hole wall of the communication tube; and a transition ring connected between the sealing ring and the end ring, a diameter of an outer periphery of the transition ring being smaller than a diameter of an inner hole of the communication tube.

Further, a first sealing structure is arranged between an outer peripheral surface of the outer tube and an inner hole wall of the communication tube, and a second sealing structure is arranged between an outer peripheral surface of the sealing tube and the inner hole wall of the communication tube.

Further, the first sealing structure is at least one of a threaded fitting seal, an interference fit seal, and an elastic fit seal.

Further, the second sealing structure includes outer threads provided on the outer peripheral surface of the sealing tube, and inner threads provided on the inner hole wall of the communication tube and matched with the outer threads. Alternatively, the second sealing structure is an inverted tapered tube body arranged on the sealing tube close to the sealing end, a maximum circumferential dimension of the inverted tapered tube body is greater than a circumferential dimension of an inner hole of the communication tube. Alternatively, the second sealing structure includes a sealing ring being sleeved on the sealing tube. An outer peripheral surface of the sealing ring is sealingly fitted with the inner hole wall of the communication tube.

Further, the communicating area is a through hole or a slit located in a periphery wall of the sealing tube, and the communicating area is capable of being blocked by an inner hole wall of the communication tube.

Further, a port of the sealing end of the sealing tube is sealed by a sealing plate.

In another aspect, a balloon is provided, and includes the self-on-off transmission assembly as described above, and a balloon body. The output end of the communication tube is connected to an opening of the balloon body.

Further, the communication tube, the outer tube, the sealing tube and the balloon body are made of polyamide, polyester, polyurethane, polyimide polyether block copolymer; polyethylene, polypropylene, polyimide, cross-linked polyethylene, cross-linked polyurethane, ionomer, polycaprolactone (PCL), polylactic acid (PLA), polylactic acid-glycolic acid copolymer (PLGA), lactide-caprolactone copolymer (PLCL), plastic starch materials, polyionic complexes, or copolymers or blends of multiple of the above materials.

In another aspect, a prosthesis for a shoulder joint is provided, and includes a balloon, a catheter, and a catheter connector that connects the balloon to the catheter. The catheter connector includes: a communication tube including an input end and an output end; an outer tube; an end of the outer tube being configured to be capable of being inserted into the communication tube from the input end and being moved out of the communication tube from the input end; and a sealing tube, configured to be capable of being placed into a tube body of the communication tube and being moved in an axial direction with respect to the tube body of the communication tube. The sealing tube includes a connecting end and a sealing end. The connecting end is configured to be detachably connected to the end of the outer tube through a connecting-driving mechanism. The sealing end is provided with a communicating area. When the communicating area is exposed to the output end, the catheter connector is in an on-state; when the communicating area is placed in the tube body of the communication tube, the catheter connector is in a sealed off-state. The outer tube is configured to at least allow the catheter connector to be switchable from being in the on-state to being in the sealed off-state, by driving the sealing tube to move along the communication tube by the connecting-driving mechanism.

### BRIEF DESCRIPTION OF THE DRAWINGS

FIG. 1 is a perspective exploded view of a self-on-off transmission assembly according to a first embodiment of the present disclosure.

FIG. 2 is a schematic view of the self-on-off transmission assembly of FIG. 1 combining with a balloon body.

FIG. 3 is a perspective exploded view of a self-on-off transmission assembly according to a second embodiment of the present disclosure.

FIG. 4 is a cross-sectional view illustrating that an outer tube, a sealing tube and a communication tube according to the second embodiment of the present disclosure are fitted and assembled.

FIG. 5 is a schematic view of the self-on-off transmission assembly of FIG. 3 combining with a balloon body.

FIG. 6 is a perspective exploded view of a self-on-off transmission assembly, according to a third embodiment of the present disclosure.

FIG. 7 is a schematic view of the self-on-off transmission assembly of FIG. 6 combining with a balloon body.

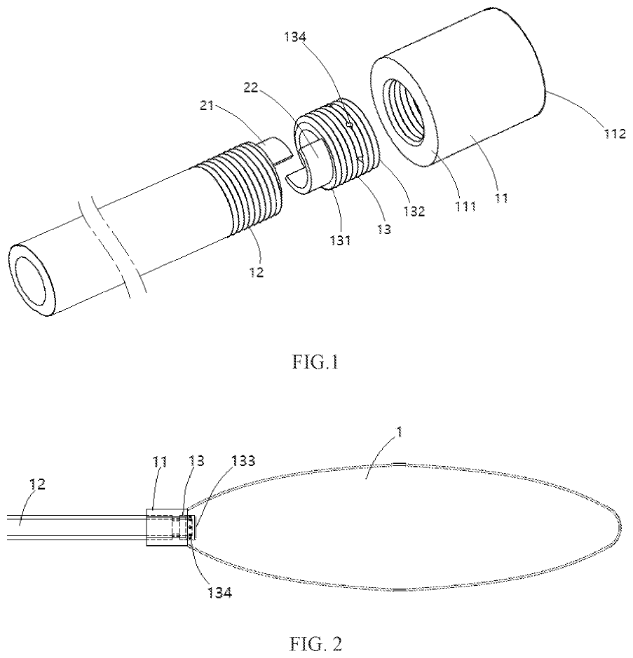

In FIGS. 1 to 2, 1—balloon body, 11—communication tube, 111—input end, 112—output end, 12—outer tube, 13—sealing tube, 131—connecting end, 132—sealing end, 133—sealing plate, 134—through hole, 21—first arc-shaped cylinder, 22—second arc-shaped cylinder,

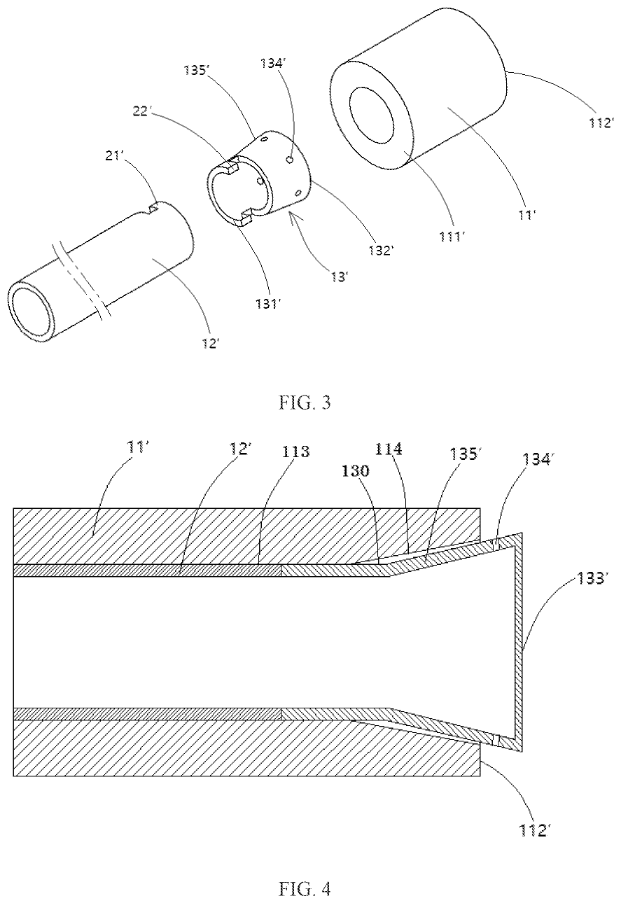

In FIGS. 3 to 5, balloon body, 11′—communication tube, 111′—input end, 112′—output end, 113—cylindrical hole, 114—tapered hole, 12′—outer tube, 13′—sealing tube, 130—cylindrical tube body, 131′—connecting end, 132′—sealing end, 133′—sealing plate, 134′—through hole, 135′—inverted tapered tube body, 21′—first buckle, 22′—second buckle,

In FIGS. 6 to 7, 1″—balloon body, 11″—communication tube, 111″—input end, 112″—output end, 12″—outer tube, 13″—sealing tube, 131″—connecting end, 132″—sealing end, 133″—sealing plate, 134″—through hole, 135″—plug-in cannula, 136—sealing ring, 137—transition ring, 138—end ring.

### DETAILED DESCRIPTION OF THE EMBODIMENTS

The present disclosure will be further described below with reference to the accompanying drawings.

In order to facilitate understanding of the present disclosure, the present disclosure will be described more fully below with reference to the accompanying drawings. Preferred embodiments of the present disclosure are shown in the accompanying drawings. However, the present disclosure may be implemented in many different forms and is not limited to the embodiments described herein. Those of ordinary skill in the art will appreciate that changes and modifications can be made to the various embodiments described herein without departing from the scope of the present disclosure, defined by the appended claims. In addition, descriptions of well-known functions and constructions may be omitted for clarity and conciseness.

It will be understood that when an element is referred to as being “fixed to” another element, it can be directly on another element or an intermediate element may also be present. When an element is referred to as being “connected to” another element, the element can be directly connected to another element or an intermediate element may be present at the same time.

Unless otherwise defined, all technical and scientific terms used herein have the same meaning as commonly understood by one of ordinary skill in the technical field to which this disclosure belongs. The terms used herein in the specification of this disclosure are for the purpose of describing specific embodiments only, and are not intended to limit this disclosure. As used herein, the term “and/or” includes any and all combinations of one or more of the associated listed items.

A balloon according to the present disclosure, such as a rotator cuff balloon, may be used as a prosthesis for reducing human soft tissue damage; and a balloon according to the present disclosure, such as a gastric balloon, may be used as a prosthesis for filling human cavities or capsular bags.

As shown in FIG. 1, a first embodiment of the present disclosure provides a self-on-off transmission assembly, which includes a communication tube 11, an outer tube 12 and a sealing tube 13. The communication tube 11 is provided with a space therein, and the outer tube 12 and the sealing tube 13 may pass through and be received in the space. The outer tube 12 is used for delivering the filling material. The communication tube 11 includes an input end 111 and an output end 112. An end of the outer tube 12 may be inserted into and moved out of the communication tube 11 from the input end 111. A first sealing structure capable of enhancing the sealing effect is arranged between an outer peripheral surface of the outer tube 12 and an inner hole wall of the communication tube 11. In the illustrated embodiment, the first sealing structure is a threaded fitting seal. For example, the outer peripheral surface of the outer tube 12 and the inner hole wall of the communication tube 11 are respectively provided with mutually matched outer threads and inner threads, so that a sealed connection between the outer tube 12 and the communication tube 11 may be realized by screwing the outer tube 12 into the communication tube 11. The sealing tube 13 is configured to be placeable into a tube body of the communication tube 11 and to be moveable in an axial direction with respect to the tube body of the communication tube. The sealing tube 13 includes a connecting end 131 and a sealing end 132. The connecting end 131 may be detachably connected to the end of the outer tube 12 through a connecting-driving mechanism. The outer peripheral surface of the sealing tube 13 and the inner hole wall of the communication tube 11 are sealingly fitted with each other by a second sealing structure. For example, the mutually matched outer threads and inner threads are respectively provided on the outer peripheral surface of the sealing tube 13 and the inner hole wall of the communication tube 11, so that the sealing tube 13 and the communication tube 11 may be sealingly connected by the thread connection. A port of the sealing end 132 is provided with a sealing plate 133, which closes the port.

The sealing end 132 is provided with a communicating area. In this embodiment, the communicating area is specifically a through hole 134 provided on a circumferential surface (peripheral wall) of the sealing tube 13 and communicating with an inner lumen and an outer wall of the sealing tube 13. It can be understood that, in other embodiments, the communicating area may be a slit in the circumferential surface of the sealing tube 13, which is not limited herein.

The through hole 134 is exposed to the output end 112 when the self-on-off transmission assembly is in an on-state, the filling material in the sealing tube 13 may flow out through the through hole 134, and the through hole 134 is in an on-state. In addition, the outer tube 12 may drive the sealing tube 13 to move from the output end 112 of the communication tube 11 to the input end 111 of the communication tube 11 by the connecting-driving mechanism until the through hole 134 is placed in the tube body of the communication tube 11, so that the through hole is switched from being in the on-state to being in a sealed off-state, while the self-on-off transmission assembly is in the sealed off-state.

When the self-on-off transmission assembly is in the on-state, the through hole 134 is exposed to the output end 112. In this case, an end of the outer tube 12 away from the sealing tube 13 may be communicated to an injection device. The filling material output from the injection device, such as liquid or gas, may be transmitted to the sealing tube 13 through the outer tube 12, and then flow out of the output end 112 of the communication tube 11 through the through holes 134 in the circumferential surface of the sealing tube 13. Since the first sealing structure and the second sealing structure have a sealing effect, the risk of leakage of the filling material between the outer tube 12, the sealing tube 13 and the communication tube 11 is reduced. When the on-state is not required, the outer tube 12 may drive the sealing tube 13 to move from the output end 112 of the communication tube 11 to the input end 111 of the communication tube 11 through the connecting-driving mechanism. When the outer tube 12 is moved to disconnect the threaded connection between the outer tube 12 and the communication tube 11, the connection with driving effect between the outer tube 12 and the sealing tube 13 can be easily released. In this case, the through hole 134 of the sealing tube 13 is placed in the tube body of the communication tube 11 and thus is sealed by the inner hole wall of the communication tube 11, and the output end 112 may be sealed by the sealing plate 133, so that the filling material located at, the input end 111 of the communication tube 11 may not flow out from the output end 112 of the communication tube 11, and the external material may not enter the communication tube 11 through the output end 112, and the self-on-off transmission assembly is in the sealed off-state.

In the above-mentioned embodiment, the connecting-driving mechanism is a circumferential coupling structure. The circumferential coupling structure includes a first member arranged at the end of the outer tube 12 facing the sealing tube 13, and a second member arranged at the connecting end 131 of the sealing tube 13. The first member and the second member are configured to be coupled with each other in a circumferential direction and decoupled from each other in the axial direction. For example, the first member is a first arc-shaped cylinder 21, the second member is a second arc-shaped cylinder 22, and the first arc-shaped cylinder 21 and the second arc-shaped cylinder 22 may abut against each other in the circumferential direction. In some embodiments, the first arc-shaped cylinder 21 and the second arc-shaped cylinder 22 may be spliced into a complete cylinder in the circumferential direction. In other embodiments, the first arc-shaped cylinder 21 and the second arc-shaped cylinder 22 may be spliced into an incomplete cylinder in the circumferential direction. When the outer tube 12 is rotated, the sealing tube 13 may be driven to rotate synchronously through the cooperation of the first arc-shaped cylinder 21 on the outer tube 12 with the second arc-shaped cylinder 22 on the sealing tube 13. While being rotated, the outer tube 12 and the sealing tube 13 may be moved from the output end 112 of the communication tube 11 to the input end 111 of the communication tube 11 through a threaded cooperation with the inner hole wall of the communication tube 11. When the outer tube 12 is moved to be completely exposed to the input end 111 of the communication tube 11, the outer tube 12 may be disconnected from the sealing tube 13 by being pulled in the axial direction. Certainly, those of ordinary skill in the art can easily replace the shapes of the first arc-shaped cylinder 21 and the second arc-shaped cylinder 22 with other shapes, for example, the first member is a protrusion, and the second member has a concave shape matched with the protrusion, as long as the first member and the second member may be coupled with each other in the circumferential direction and decoupled from each other in the axial direction.

In the first embodiment, six through holes 134 are provided, for example. The plurality of through holes 134 are convenient to improve the speed of material transmission. In some other embodiments, the number of the through holes 134 may be set as needed, which is not limited herein.

The balloon shown in FIG. 2 may be Obtained by arranging the self-on-off transmission assembly on the balloon body 1. In FIG. 2, the output end 112 of the communication tube 11 is connected to an opening of the balloon body 1. The outer tube 12 is pre-inserted into the communication tube 11 and kept coupled with the sealing tube 13 in the circumferential direction. The balloon body 1 is folded in a puncture cannula, delivered to a preset part of the human body and released under direct vision of an endoscope (e.g., an arthroscope). In this case, the self-on-off transmission assembly is in the on-state, and the balloon body 1 may be filled by injecting physiological saline through the outer tube 12, and then the outer tube 12 is rotated, and the outer tube 12 may drive the sealing tube 13 to rotate synchronously and to move from the output end 112 of the communication tube 11 to the input end 111 of the communication tube 11. When the outer tube 12 is moved to be completely exposed to the input end 111 of the communication tube 11, the outer tube 12 may be pulled in the axial direction to disconnect the outer tube 12 from the sealing tube 13. In this case, the through hole 134 is placed in the tube body of the communication tube 11 and thus is sealed by the inner hole wall of the communication tube 11, and the self-on-off transmission assembly is in the sealed off-state, the input end 111 of the communication tube 11 and the output end 112 of the communication tube 11 are not in communication with each other, and the physiological saline in the balloon body 1 may not flow out of the opening.

According to the balloon of the first embodiment, the separation and withdrawal of the outer tube 12 and the sealing of the balloon body 1 may be performed simultaneously. While the outer tube 12 is disconnected from the sealing tube 13, the through holes 134 of the sealing tube 13 are moved into the tube body of the communication tube 11 so as to be blocked, so that the self-on-off transmission assembly is in the sealed off-state. The opening of the balloon body 1 may be directly sealed by the sealing plate 133, and the sealing effect is good.

### Second Embodiment

A second embodiment shown in FIG. 3 to FIG. 5 provides another self-on-off transmission assembly and a balloon, the main structure of which is generally similar to those described in the first embodiment, and the main difference from those described in the first embodiment lies in structural forms of the first sealing structure, the second sealing structure and the connecting-driving mechanism. The first sealing structure between the outer peripheral surface of the outer tube 12′ and the inner hole wall of the communication tube 11′ is an interference fit seal. Specifically, the outer tube 12′ is inserted into the communication tube 11′ from the input end 111′, and the outer peripheral surface of the outer tube 12′ is in interference fit with the inner hole wall of the communication tube 11′ to achieve sealing.

In addition, in the second embodiment, the second sealing structure between the outer peripheral surface of the sealing tube 13′ and the inner hole wall of the communication tube 11′ is an inverted tapered tube body 135′ arranged on the sealing tube 13′ close to the sealing end 132′. The inverted tapered tube body 135′ may be in an interference fit with the inner hole wall of the communication tube 11′. Referring to FIG. 4, the sealing tube 13′ includes a cylindrical tube body 130 connected with the inverted tapered tube body 135′. The cylindrical tube body 130 is used to be detachably connected to an end of the outer tube 12′ through the connecting-driving mechanism. The through hole 134′ is provided on the peripheral wall of the inverted tapered tube body 135′. The circumferential dimension of the inverted tapered tube body 135′ gradually increases in an axial direction away from the cylindrical tube body 130. An inner hole of the communication tube 11′ includes a cylindrical hole 113. The cylindrical tube body 130 of the sealing tube 13′ is slidably matched with the cylindrical hole 113. When the outer tube 12′ drives the sealing tube 13′ to move from an output end 112′ of the communication tube 11′ to an input end 111′ of the communication tube 11′ along the cylindrical hole 113 through the connecting-driving mechanism, the outer peripheral surface of the inverted tapered tube body 135′ may be abutted on the inner hole wall of the communication tube 11′, so as to realize the sealed off-state of the through hole 134′. In particular, when the self-on-off transmission assembly is applied into the balloon, the balloon body 1′ implanted into the predetermined part is subjected to a pressure, and then the filling liquid in the balloon body exerts a pressure on the sealing plate 133′, so that the connection between the inverted tapered tube body 135′ and the communication tube 11′ is tighter. The interference fit has good stability and good sealing effect. Preferably, the sealing tube 13′ is made of polyamide and is integrally formed. The communication tube 11′ is made of polyethylene material. The sealing tube 13′ and the communication tube 11′ are flexible and deformable, so that it is easier to operate the outer tube 12′ to drive the sealing tube 13′ to move from the output end 112′ of the communication tube 11′ to the input end 111′ of the communication tube 11′, and the sealing performance due to the close contact between the outer tube 12′ and the communication tube 11′ is better.

In this embodiment, the inner hole of the communication tube 11′ has a tapered hole 114 matched with the inverted tapered tube body 135′. The tapered hole 114 is connected to the cylindrical hole 113, and the tapered hole 114 is close to the output end 112′. When the outer tube 12′ drives the sealing tube 13′ to move from the output end 112′ of the communication tube 11′ to the input end 111′ of the communication tube 11′ through the connecting-driving mechanism, the outer peripheral surface of the inverted tapered tube body 135′ may be just fitted and embedded into the tapered hole 114 of the communication tube 11′, so as to achieve the sealed off-state of the through hole 134′. However, in other embodiments, the through hole 134′ may be provided on the cylindrical tube body 130, the inner hole of the communication tube 11′ may be the cylindrical hole and no tapered hole 114 is provided. When the through hole 134′ is moved to be closed by the inner hole wall of the communication tube 11′, the inverted tapered tube body 135′ is abutted against a port of the inner hole of the communication tube 11′, and the sealed off-state of the through hole 134′ may also be achieved.

In the second embodiment, the connecting-driving mechanism includes an axial and circumferential coupling structure. The axial and circumferential coupling structure includes a third member arranged at an end of the outer tube 12′ facing the sealing tube 13′, and a fourth member arranged at a connecting end 131′ of the sealing tube 13. The third member and the fourth member may be coupled with and decoupled from each other in the axial and circumferential directions. In FIG. 3, the third member is a first buckle 21′, the fourth member is a second buckle 22′. The first buckle 21′ and the second buckle 22′ may be engaged with each other. In this embodiment, the first buckle 21′ are two hooks arranged at the end of the outer tube 12′ and extending in the circumferential direction, and the second buckle 22′ are two hooks arranged at the end of the sealing tube 13′ and extending in the circumferential direction. In some embodiments, the first buckle 21′ and the second buckle 22′ may be engaged to form a circle in the circumferential direction. In FIG. 3, the two hooks of the first buckle 21′ at the end of the outer tube 12′ are arranged opposite to each other in the circumferential direction, and the two hooks of the second buckle 22′ at the end of the sealing tube 13′ are also arranged opposite to each other in the circumferential direction, and correspond to the directions of the two hooks of the first buckle 21′, respectively. During assembly, the first buckle 21′ of the outer tube 12′ can pass over the second buckle 22′ of the sealing tube 13′ in the axial direction, and the first buckle 21′ of the outer tube 12′ and the second buckle 22′ of the sealing tube 13′ move toward each other in the radial direction to be hooked and engaged with each other by means of concave-convex fit, so as to be connected with each other to form a circle in the circumferential direction. In some other embodiments, the two hooks of the first buckle 21′ at the end of the outer tube 12′ are arranged in the same direction in the circumferential direction (for example, both are arranged clockwise), and the two hooks of the second buckle 22′ at the end of the sealing tube 13′ are also arranged in the same direction in the circumferential direction, and correspond to the directions of the two hooks of the first buckle 21′, respectively (for example, both are arranged counterclockwise). During assembly, the outer tube 12′ and the sealing tube 13′ are rotated oppositely in the circumferential direction, so that any hook of the first buckle 21′ is hooked and engaged with (snapped to) a corresponding hook of the second buckle 22′ by means of concave-convex fit. Then, the sealing tube 13′ may be pulled in the axial direction by pulling the outer tube 12′ in the axial direction, so that the sealing tube 13′ is moved from the output end 112′ of the communication tube 11′ to the input end 111′ of the communication tube 11′, which allows the sealing tube 13′ to block the communication tube 11′. When the sealing tube 13′ is pulled in the axial direction to block the communication tube 11′, the outer tube 12′ and the sealing tube 13′ may be rotated synchronously in the circumferential direction to increase the moving speed, so that the operation of blocking the communication tube 11′ is more flexible and convenient for clinical application. Certainly, those of ordinary skill in the art can easily replace the first buckle 21′ and the second buckle 22′ with other structures, such as two locking teeth connected and matched with each other.

The sealing tube 13′ and the outer tube 12′ according to the second embodiment are snap-connected together and placed in the communication tube 11′, and the self-on-off transmission assembly is arranged in the opening of the balloon body 1′, thereby obtaining a balloon as shown in FIG. 5. The balloon body 1′ is folded inside the puncture cannula, delivered to a predetermined part of the human body and released. In this case, the self-on-off transmission assembly is in the on-state, and the balloon body 1′ is filled by injecting physiological saline through the outer tube 12′. Since the first sealing structure and the second sealing structure have a sealing effect, the filling material may not be seeped out between the outer tube 12′ and the communication tube 11′, and between the sealing tube 13′ and the communication tube 11′. When the balloon body 1′ is required to be sealed, the outer tube 12′ and the sealing tube 13′ are pulled in the axial direction, so that the outer tube 12′ and the sealing tube 13′ move from the output end 112′ of the communication tube 11′ to the input end 111′ of the communication tube 11′. When the outer tube 12′ is completely exposed to the input end 111′, the outer tube 12′ and the sealing tube 13′ are rotated oppositely in the circumferential direction, or the outer tube 12′ are directly moved in the radial direction. As a result, the circumferential coupling between the hooks of the first buckle 21′ and the hooks of the second buckle 22′ may be decoupled. The outer tube 12′ is pulled in the axial direction, the outer tube 12′ can be completely detached from the communication tube 11′ in the axial direction, while the through hole 134′ is placed in the tube body of the communication tube 11′ and is blocked by the inner hole wall of the communication tube 11′. In this case, the self-on-off transmission assembly is in a sealed off-state, and the input end 111′ and the output end 112′ of the communication tube 11′ are not in communication with each other, and the physiological saline in the balloon body 1′ does not overflow the opening.

### Third Embodiment

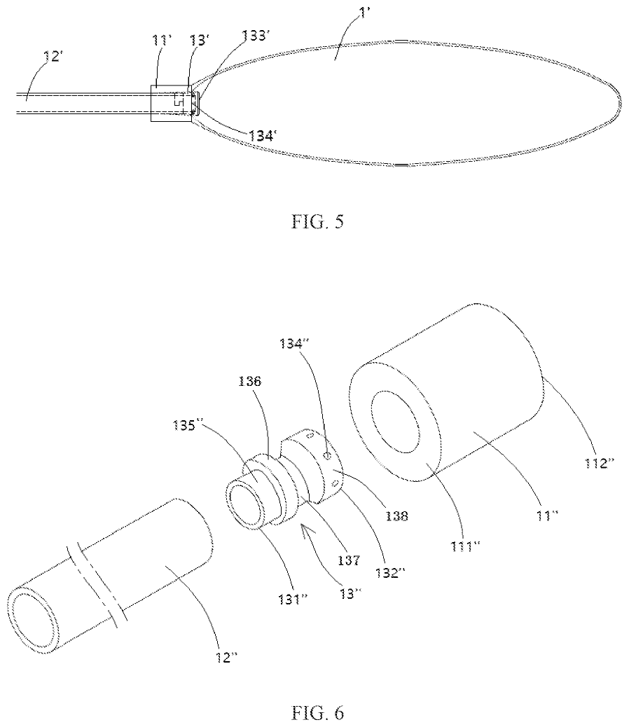

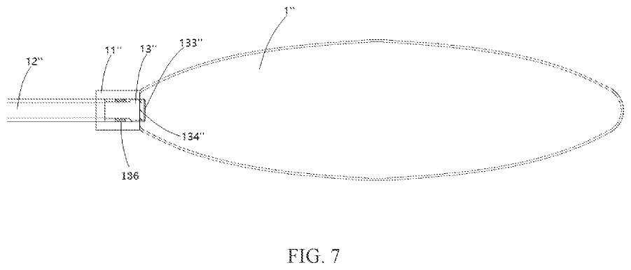

The third embodiment shown in FIG. 6 and FIG. 7 provides yet another self-on-off transmission assembly and a balloon. In this embodiment, the main structure is generally similar to those described in the first embodiment, and the main difference from those described in the first embodiment lies in structural forms of the first sealing structure, the second sealing structure and the connecting-driving mechanism. The first sealing structure between the outer peripheral surface of the outer tube 12″ and the inner hole wall of the communication tube 11″ is an elastic fit seal. For example, the outer tube 12″ is inserted into the communication tube 11″ from the input end 111″, and the outer peripheral surface of the outer tube 12″ is cooperated elastically with the inner hole wall of the communication tube 11″, and thus the inner hole wall of the communication tube 11″ is pressed against the outer peripheral surface of the outer tube 12″ to achieve sealing.

In addition, in the third embodiment, the second sealing structure between the outer peripheral surface of the sealing tube 13″ and the inner hole wall of the communication tube 11″ includes a sealing ring 136 arranged on the outer periphery of the sealing tube 13″. An outer peripheral surface of the sealing ring 136″ is sealingly fitted with the inner hole wall of the communication tube 11″. For example, the outer peripheral surface of the sealing ring 136 is in close contact with the inner hole wall of the communication tube 11″, which may prevent the filling material from flowing to the input end 111″ from the output end 112″ of the communication tube 11″ through a gap between the outer peripheral surface of the outer tube 12″ and the inner hole walls of the communication tube 11″.

The connecting-driving mechanism in the third embodiment includes an elastic coupling structure. The elastic coupling structure includes a plug-in cannula 135″ arranged at a connecting end 131″ of the sealing tube 13″. The plug-in cannula 135″ may be inserted into the inner hole of the end of the outer tube 12″. When the outer tube 12″ and the sealing tube 13″ are placed in the communication tube 11″, the end of the outer tube 12″ is clamped between the communication tube 11″ and the plug-in cannula 135″, The inner hole wall of the communication tube 11″ exerts a pressing force on the outer tube 12″ in the radial direction, and the pressing force is transmitted to the plug-in cannula 135″ in the radial direction. In this case, there is a large static friction force between the inner hole wall of the outer tube 12″ and the outer peripheral surface of the plug-in cannula 135″. When the outer tube 12″ is pulled, the static friction force between the outer tube 12″ and the plug-in cannula. 135″ allows the sealing tube 13″ to be pulled synchronously in the axial direction, so that the sealing tube 13″ is moved from the output end 112″ of the communication tube 11″ to the input end 111″ of the communication tube 11″, and thus the operation of blocking the communication tube 11″ is simple and convenient for clinical application. In some embodiments, the communication tube 11″, the sealing tube 13″, and the outer tube 12″ may be made of polyurethane, which has strong deformation ability. The outer tube 12″ may be deformed when being subjected to the pressing force from the inner hole wall of the communication tube 11″, which is beneficial for the pressing force to be transmitted to the plug-in cannula 135″ in the radial direction, thereby increasing the static friction force between the inner hole wall of the outer tube 12″ and the outer periphery surface of the plug-in cannula 135″, causing the sealing tube 13″ to be pulled more easily.

As can be seen from FIG. 6, the plug-in cannula 135″ is connected to the sealing ring 136, and the sealing tube 13″ further includes a transition ring 137 and an end ring 138. The transition ring 137 is connected between the sealing ring 136 and the end ring 138, and an diameter of the outer periphery the transition ring 137 is smaller than a hole diameter of the inner hole of the communication tube 11″ The through hole 134″ is arranged on a peripheral wall of the end ring 138, and an outer peripheral surface of the end ring 138 may be in elastic sealing fit with the inner hole wall of the communication tube 11″.

The self-on-off transmission assembly shown in FIG. 6 is connected to the balloon body 1″ to obtain a balloon as shown in FIG. 7. When the self-on-off transmission assembly is in the on-state, since the first sealing structure and the second sealing structure have a sealing effect, the physiological saline filled into the balloon body 1″ does not seep out between the outer tube 12″ and the communication tube 11″, and between the sealing tube 13″ and the communication tube 11″. When the outer tube 12″ and the sealing tube 13″ are placed in the communication tube 11″, the inner hole wall of the communication tube 11″ exerts a pressing force on the outer tube 12″ in the radial direction, so that a large static friction force is generated between the inner hole wall of the tube body of the outer tube 12″ and the outer peripheral surface of the plug-in cannula 135″. When the balloon body 1″ is required to be sealed, the outer tube 12″ is pulled in the axial direction, and the static friction force between the outer tube 12″ and the plug-in cannula 135″ allows the sealing tube 13″ to be pulled synchronously in the axial direction, so that the sealing tube 13″ is moved from the output end 112″ to the input end 111″ of the communication tube 11″. When the outer tube 12″ is completely exposed to the communication tube 11″, the circumferential pressing force acting on the outer tube 12″ by the inner hole wall of the communication tube 11″ disappears, and the static friction force between the inner hole wall of the tube body of the outer tube 12″ and the outer peripheral surface of the plug-in cannula 135″ is reduced, so that the outer tube 12″ may be easily separated from the sealing tube 13″. In this case, the through hole 134″ is placed in the tube body of the communication tube 11″ and is blocked by the inner hole wall of the communication tube 11″, and the output end 112″ of the communication tube 11″ is sealed by the sealing plate 133″ of the sealing end 132″. Thus, the self-on-off transmission assembly is in a sealed off-state, the input end 111″ and the output end 112″ of the communication tube 11″ are not in communication with each other, and the physiological saline in the balloon body 1″ may not overflow the opening.

In practical applications, the self-on-off transmission assembly described in the above embodiments can be directly used as a catheter connection product applied in a prosthesis of human joints, soft tissues, cavities or capsular bags. For example, the prosthesis includes a balloon, a catheter, and a catheter connector that connects the balloon to the catheter. A structure of the catheter connector is substantially the same as that of the above-mentioned self-on-off transmission assembly. When the prosthesis is implanted into the human body and the catheter connector is in an on-state, a filling medium may be injected into the balloon through the catheter. After filling, the catheter connector is operated to be in a sealed off-state. In this case, the balloon is in a sealed state, which may play the role of stable support and lubrication to ensure smooth and frictionless movement between bones, and help the organism restore the characteristics of the joint by replacing the damaged joint capsule or providing temporary support.

Any one of the communication tube, the outer tube, the sealing tube and the balloon body listed in the above embodiments is made of, but is not limited to be made of, polyamide, polyester, polyurethane, polyimide: polyether block copolymer, polyethylene, polypropylene, polyimide, cross-linked polyethylene, cross-linked polyurethane, ionomer, polycaprolactone (PCL), polylactic acid (PLA), polylactic acid-glycolic acid copolymer (PLGA), lactide-caprolactone copolymer (PLCL), plastic starch materials, polyionic complexes, or copolymers or blends thereof. These materials involve biocompatible or biodegradable materials, which can be optimally matched according to the application environments. For example, if the implantable balloon is needed to replace organs in the organism, the biocompatible material is preferred; and if the balloon implantable is to provide an auxiliary function in the recovery period after the injury of the organism, the biodegradable material is preferred.

It can be understood that the connecting-driving mechanism, the first sealing structure, and the second sealing structure described in the above embodiments can be freely combined and matched in other embodiments as required. For example, the connecting-driving mechanism described in the second embodiment can also be directly used in the first embodiment, which does not affect the implementation of this disclosure and is not limited herein.

The technical features of the aforementioned embodiments can be combined arbitrarily. In order to simply the description, all possible combinations of the technical features in the aforementioned embodiments are not described. However, as long as there is no contradiction in the combinations of these technical features, they should be considered to be fallen into the scope described in the present specification.

Only several implementations of the present disclosure are illustrated in the aforementioned embodiments, and the description thereof is relatively specific and detailed, but is should not be understood as a limitation on the scope of the present disclosure. It should be noted that for those of ordinary skill in the art, without departing from the concept of the present disclosure, several modifications and improvements can be made, which all fall within the protection scope of the present disclosure. Therefore, the protection scope of the present disclosure shall be subject to the appended claims.

1. A self-on-off transmission assembly, comprising:
a communication tube, comprising an input end and an output end;
an outer tube, an end of the outer tube being configured to be capable of being inserted into the communication tube from the input end and being moved out of the communication tube from the input end; and
a sealing tube, configured to be capable of being placed into a tube body of the communication tube and being moved in an axial direction with respect to the tube body of the communication tube; the sealing tube comprising a connecting end and a sealing end; the connecting end being configured to be detachably connected to the end of the outer tube through a connecting-driving mechanism; the sealing end being provided with a communicating area;
wherein when the communicating area is exposed to the output end, the self-on-off transmission assembly is in an on-state; when the communicating area is placed in the tube body of the communication tube, the self-on-off transmission assembly is in a sealed off-state; and
wherein the outer tube is configured to at least allow the self-on-off transmission assembly to be switchable from being in the on-state to being in the sealed off-state, by driving the sealing tube to move along the communication tube by the connecting-driving mechanism.

2. The self-on-off transmission assembly of claim 1, wherein the connecting-driving mechanism comprises a first member arranged at the end of the outer tube, and a second member arranged at the connecting end of the sealing tube, and the first member and the second member are configured to be capable of being coupled with each other in a circumferential direction and decoupled from each other in the axial direction.

3. The self-on-off transmission assembly of claim 1, wherein the connecting-driving mechanism comprises a third member arranged at the end of the outer tube, and a fourth member arranged at the connecting end of the sealing tube, and the third member and the fourth member are configured to be capable of being coupled with each other in the axial direction and in a circumferential direction, and being decoupled from each other in the axial direction and in the circumferential direction.

4. The self-on-off transmission assembly of claim 1, wherein the connecting-driving mechanism comprises a plug-in cannula arranged at the connecting end of the sealing tube; the plug-in cannula is configured to be capable of being inserted into an inner hole of the end of the outer tube; when the end of the outer tube is inserted into the communication tube, the end of the outer tube is clamped between the communication tube and the plug-in cannula.

5. The self-on-off transmission assembly of claim 2, wherein the first member comprises a first arc-shaped cylinder, the second member comprises a second arc-shaped cylinder, and the first arc-shaped cylinder and the second arc-shaped cylinder are capable of being abutted against each other in the circumferential direction.

6. The self-on-off transmission assembly of claim 5, wherein an outer peripheral wall of the end of the outer tube and an outer peripheral wall of the sealing tube are both provided with outer threads; and an inner hole wall of the communication tube is provided with inner threads matched with the outer threads.

7. The self-on-off transmission assembly of claim 3, wherein the third member is a first buckle, the fourth member is a second buckle, and the first buckle and the second buckle are capable of being engaged with each other.

8. The self-on-off transmission assembly of claim 7, wherein the first buckle and the second buckle are hooks extending in the circumferential direction.

9. The self-on-off transmission assembly of claim 3, wherein an inner hole of the communication tube comprises a cylindrical hole and a tapered hole which are connected to each other, the tapered hole is close to the output end, the sealing tube comprises a cylindrical tube body and an inverted tapered tube body which are connected to each other, the fourth member is arranged on the cylindrical tube body, the communicating area is arranged in the inverted tapered tube body, the cylindrical tube body is slidably matched with the cylindrical hole, and the inverted tapered tube body is adapted to be abutted against the tapered hole.

10. The self-on-off transmission assembly of claim 3, wherein the sealing tube comprises a cylindrical tube body and an inverted tapered tube body which are connected to each other, the fourth member and the communicating area are arranged in the cylindrical tube body, the cylindrical tube body is slidably matched with an inner hole of the communication tube, and the inverted tapered tube body is adapted to be abutted against an end of the communication tube.

11. The self-on-off transmission assembly of claim 4, wherein the sealing tube comprises: a sealing ring connected to the plug-in cannula, an outer periphery surface of the sealing ring being sealingly fitted with an inner hole wall of the communication tube; an end ring, the communicating area being arranged on an periphery wall of the end ring, an outer periphery surface of the end ring being capable of being sealingly fitted with the inner hole wall of the communication tube; and a transition ring connected between the sealing ring and the end ring, a diameter of an outer periphery of the transition ring being smaller than a diameter of an inner hole of the communication tube.

12. The self-on-off transmission assembly of claim 1, wherein a first sealing structure is arranged between an outer peripheral surface of the outer tube and an inner hole wall of the communication tube, and a second sealing structure is arranged between an outer peripheral surface of the sealing tube and the inner hole wall of the communication tube.

13. The self-on-off transmission assembly of claim 12, wherein the first sealing structure is at least one of a threaded fitting seal, an interference fit seal, and an elastic fit seal.

14. The self-on-off transmission assembly of claim 12, wherein the second sealing structure comprises outer threads provided on the outer peripheral surface of the sealing tube, and inner threads provided on the inner hole wall of the communication tube and matched with the outer threads; or the second sealing structure is an inverted tapered tube body arranged on the sealing tube close to the sealing end, a maximum circumferential dimension of the inverted tapered tube body is greater than a circumferential dimension of an inner hole of the communication tube; or the second sealing structure comprises a sealing ring being sleeved on the sealing tube, an outer peripheral surface of the sealing ring is sealingly fitted with the inner hole wall of the communication tube.

15. The self-on-off transmission assembly of claim 1, wherein the communicating area is a through hole or a slit located in a periphery wall of the sealing tube, and the communicating area is capable of being blocked by an inner hole wall of the communication tube.

16. The self-on-off transmission assembly of claim 15, wherein a port of the sealing end of the sealing tube is sealed by a sealing plate.

17. A balloon, comprising:
the self-on-off transmission assembly of claim 1; and
a balloon body;
wherein the output end of the communication tube is connected to an opening of the balloon body.

18. The balloon of claim 17, wherein the communication tube, the outer tube, the sealing tube and the balloon body are made of polyamide, polyester, polyurethane, polyamide polyether block copolymer, polyethylene, polypropylene, polyimide, cross-linked polyethylene, cross-linked polyurethane, ionomer, polycaprolactone (PCL), polylactic acid (PLA), polylactic acid-glycolic acid copolymer (PLGA), lactide-caprolactone copolymer (PLCL), plastic starch materials, polyionic complexes, or copolymers or blends thereof.

19. A prosthesis for a shoulder joint, comprising a balloon, a catheter, and a catheter connector that connects the balloon to the catheter, wherein the catheter connector comprises:
a communication tube comprising an input end and an output end;
an outer tube, an end of the outer tube being configured to be capable of being inserted into the communication tube from the input end and being moved out of the communication tube from the input end; and
a sealing tube configured to be capable of being placed into a tube body of the communication tube and being moved in an axial direction with respect to the tube body of the communication tube, the sealing tube comprising a connecting end and a sealing end, the connecting end being configured to be detachably connected to the end of the outer tube through a connecting-driving mechanism, and the sealing end being provided with a communicating area,
wherein when the communicating area is exposed to the output end, the catheter connector is in an on-state; when the communicating area is placed in the tube body of the communication tube, the catheter connector is in a sealed off-state; and
wherein the outer tube is configured to at least allow the catheter connector to be switchable from being in the on-state to being in the sealed off-state, by driving the sealing tube to move along the communication tube by the connecting-driving mechanism.
